(19) United States
(12) Patent Application Publication
(10) Pub. No.: US 2017/0304657 A1
(43) Pub. Date: Oct. 26, 2017

(54) COMPOSITION FOR IMPROVING SKIN TRANSPARENCY CONTAINING BLUE COMPLEX POWDER

(71) Applicant: AMOREPACIFIC CORPORATION, Seoul (KR)

(72) Inventors: Se Jun PARK, Yongin-si, Gyeonggi-do (KR); Choon Bok JEONG, Yongin-si, Gyeonggi-do (KR); Myeong Ryeol LEE, Yongin-si, Gyeonggi-do (KR); Sun Young PARK, Yongin-si, Gyeonggi-do (KR)

(21) Appl. No.: 15/520,626

(22) PCT Filed: Jul. 20, 2015
(86) PCT No.: PCT/KR2015/007493

§ 371 Date: Apr. 20, 2017

(30) Foreign Application Priority Data
Oct. 23, 2014 (KR) 10-2014-0144517

(51) Int. Cl. A61Q 1/02; A61K 8/25; A61K 8/29; A61Q 19/00

(52) U.S. Cl. CPC A61Q 1/02; A61Q 19/00; A61K 8/25; A61K 8/29; A61K 2800/436

(57) ABSTRACT

The present disclosure relates to a blue complex powder including a flat-shaped fine powder and a coating layer coated on the flat-shaped fine powder and a composition for improving skin transparency which includes the blue complex powder as an active ingredient. The complex powder may provide improved naturalness, gloss and transparency by reflecting, emitting or amplifying blue light. In addition, the complex powder may provide uniform applicability on skin and uniform interference by restricting the particle size distribution of the complex powder to 1-16 μm. The complex powder is useful because it may be used in a cosmetic composition for base makeup.

### TECHNICAL FIELD

The present disclosure relates to a composition for improving skin transparency, which contains a blue complex powder as an active ingredient.

### BACKGROUND ART

Cosmetics are largely classified into base makeup cosmetics which are applied onto the entire face and point makeup cosmetics which are applied onto only a small portion of the face. The base makeup cosmetics are used mainly to express clear skin by covering freckles, wrinkles, moles, etc. of the face.

Recently, as natural-looking makeup is favored, a transparent powder material is used, the covering effect of cosmetics is reduced, a fine powder of jewelry is used or a pearl powder is used to provide transparent skin expression with minimum amount of makeup cosmetics.

However, simply with use of a transparent material or reduction of the covering effect of cosmetics, the primary goal of makeup, i.e. covering, cannot be satisfied. And, when a fine powder of jewelry is used, the intrinsic transparency of the jewelry is lost during the manufacturing or pulverization process.

The method of using a pearl powder is favored the most. When the pearl powder is applied being contained in a cosmetic composition, applicability, uniformity and shapability are unsatisfactory because the powder has varying sizes and a glittering effect occurs because of nonuniform interference. To overcome these disadvantages, there has been an attempt to reduce the particle size of the pearl powder and achieve a narrow particle distribution. However, this attempt was not successful due to turbidity.

### REFERENCES OF RELATED ART

### Patent Document

(Patent document 1) KR1993-0021184 A

### DISCLOSURE

### Technical Problem

In an aspect, the present disclosure is directed to providing a composition for improving skin transparency.

In another aspect, the present disclosure is directed to providing a cosmetic composition which provides improved naturalness, gloss and transparency.

In another aspect, the present disclosure is directed to providing a cosmetic composition which has improved applicability and uniformity.

In another aspect, the present disclosure is directed to providing a cosmetic composition for base makeup which provides improved naturalness, gloss, transparency, applicability and uniformity.

### Technical Solution

In an aspect, the present disclosure provides a composition for improving skin transparency, which contains a blue composite powder as an active ingredient, wherein the complex powder includes a flat-shaped fine powder and a coating layer coated on the flat-shaped fine powder, the flat-shaped fine powder is one or more selected from a group consisting of mica, synthetic mica, alumina, borosilicate, talc and sericite, the coating layer includes one or more selected from a group consisting of titanium dioxide and tin oxide, and 90% or more of the particle constituting the complex powder has a size within a range of 1-16 μm.

### Advantageous Effects

In an aspect, a composition for improving skin transparency of the present disclosure provides natural luster and transparency when applied by reflecting, emitting or amplifying blue light and may provide uniform applicability on skin and uniform interference by restricting the particle size distribution of a complex powder to 1-16 μm. Accordingly, it may be used as a cosmetic composition such as a cosmetic composition for base makeup.

### BRIEF DESCRIPTION OF DRAWINGS

FIG. 1 shows blue light reflection spectra (FSB: blue complex powder included (Example 1), Normal: normal (Comparative Example 1), FB: blue pigment included (Comparative Example 3), ordinate: reflectivity).

### BEST MODE

Hereinafter, the present disclosure is described in detail.

In an aspect, the present disclosure provides a composition for improving skin transparency, which contains a blue composite powder as an active ingredient, wherein the complex powder includes a flat-shaped fine powder and a coating layer coated on the flat-shaped fine powder, the flat-shaped fine powder is one or more selected from a group consisting of mica, synthetic mica, alumina, borosilicate, talc and sericite, the coating layer includes one or more selected from a group consisting of titanium dioxide and tin oxide, and 90% or more of the particle constituting the complex powder has a size within a range of 1-16 μm.

In another aspect, the present disclosure provides a method for improving skin transparency of a subject, which includes administering an effective amount of a composition containing a blue complex powder as an active ingredient to the subject, wherein the complex powder includes a flat-shaped fine powder and a coating layer coated on the flat-shaped fine powder, the flat-shaped fine powder is one or more selected from a group consisting of mica, synthetic mica, alumina, borosilicate, talc and sericite, the coating layer includes one or more selected from a group consisting of titanium dioxide and tin oxide, and 90% or more of the particle constituting the complex powder has a size within a range of 1-16 μm.

In another aspect, the present disclosure provides a use of a blue complex powder for preparing a composition for improving skin transparency, wherein the complex powder includes a flat-shaped fine powder and a coating layer coated on the flat-shaped fine powder, the flat-shaped fine powder is one or more selected from a group consisting of mica, synthetic mica, alumina, borosilicate, talc and sericite, the coating layer includes one or more selected from a group consisting of titanium dioxide and tin oxide, and 90% or more of the particle constituting the complex powder has a size within a range of 1-16 μm.

In another aspect, the present disclosure provides a use of a composition containing a blue complex powder as an active ingredient for improving skin transparency, wherein the complex powder includes a flat-shaped fine powder and a coating layer coated on the flat-shaped fine powder, the flat-shaped fine powder is one or more selected from a group consisting of mica, synthetic mica, alumina, borosilicate, talc and sericite, the coating layer includes one or more selected from a group consisting of titanium dioxide and tin oxide, and 90% or more of the particle constituting the complex powder has a size within a range of 1-16 μm.

In the present disclosure, “flat-shaped” may mean the flap shape of a fine powder. For example, it may mean a shape which is relatively thin horizontally, such as a plate, and may include sheet, scale, lamina and needle shapes.

In this aspect, the flat-shaped fine powder may be one or more selected from a group consisting of mica, synthetic mica, alumina, borosilicate, talc and sericite. However, without being limited thereto, it may also be one or more selected from a group consisting of kaolin, calcium carbonate, barium sulfate, diatomite, magnesium carbonate, magnesium silicate, barium carbonate, boron nitride, hydroxyapatite, zirconium oxide, titanium-coated mica, sodium borosilicate and calcium borosilicate

In the present disclosure, a “complex powder” may mean a powder in which a coating layer has been formed on the surface of a fine powder.

In the present disclosure, a “particle size distribution” may mean a range of particle diameter to which the particle with the smallest particle diameter and the particle with the largest particle diameter among the individual particles constituting a complex powder belong. In other words, it means range of particle diameter to which all the individual particles constituting the complex powder belong. For example, the particle size distribution may be measured using Matersizer 2000 available from Malvern.

In this aspect, the particle size distribution of the complex powder may be 1-16 μm. Specifically, the minimum value of the particle size may be 1 μm, 2 μm, 3 μm, 4 μm, 5 μm, 6 μm, 7 μm, 8 μm, 9 μm, 10 μm, 12 μm, 14 μm or 16 μm, and the maximum value of the particle size may be 16 μm, 15 μm, 14 μm, 13 μm, 12 μm, 11 μm, 10 μm, 8 μm, 6 μm, 4 μm, 2 μm or 1 μm.

In this aspect, when the size of the complex powder increases (e.g., when it is greater than 17 μm), the powder may be difficult to be uniformly applied on skin and a uniform interference effect may not be achieved. And, when the size of the complex powder falls outside the range of 1-16 μm, unnatural turbidity may occur due to light scattering.

In this aspect, the number of the particles having sizes belonging to the range of 1-16 μm may be 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more or 99.9% or more based on the total number of the particles of the complex powder, although not being limited thereto.

In this aspect, the blue complex powder may be reflect blue visible light having a wavelength of 390-500 nm, specifically blue light having a wavelength of 400-450 nm, although not being limited thereto. Specifically, the blue light may have a wavelength of 390 nm or longer, 400 nm or longer, 410 nm or longer, 420 nm or longer, 430 nm or longer, 440 nm or longer, 450 nm or longer, 460 nm or longer, 470 nm or longer, 480 nm or longer, 490 nm or longer or 500 nm or longer, and 500 nm or shorter, 490 nm or shorter, 480 nm or shorter, 470 nm or shorter, 460 nm or shorter, 450 nm or shorter, 440 nm or shorter, 430 nm or shorter, 420 nm or shorter, 410 nm or shorter or 400 nm or shorter.

In the present disclosure, a “coating layer” may mean a layer formed as a coating agent is fused or bound on the surface of a fine powder by a known method including mechanical or chemical fusion.

In the composition for improving skin transparency according to an aspect of the present disclosure, the coating layer may include one or more selected from a group consisting of titanium dioxide and tin oxide, although not being limited thereto. The titanium dioxide and tin oxide may be in the form of a powder, a nanopowder or a liquid or a gel containing a powder, although not being limited thereto.

In this aspect, the coating layer may be included in an amount of 2-60 wt % based on the total weight of the complex powder. Specifically, it may be included in an amount of 2 wt % or more, 10 wt % or more, 20 wt % or more, 30 wt % or more, 38 wt % or more, 45 wt % or more, 50 wt % or more, 55 wt % or more or 60 wt % or more, and 60 wt % or less, 55 wt % or less, 49 wt % or less, 44 wt % or less, 39 wt % or less, 34 wt % or less, 29 wt % or less, 21 wt % or less, 15 wt % or less, 9 wt % or less, 5 wt % or less or 2 wt % or less, although not being limited thereto. When the amount of the coating layer belongs to the above range, blue light may be reflected, scattered or amplified such that naturalness, gloss and skin transparency can be expressed sufficiently.

The composition for improving skin transparency according to an aspect of the present disclosure may include 5-40 wt % of the blue complex powder based on the total weight of the composition, although not being limited thereto. Specifically, the blue complex powder may be included in an amount of 5 wt % or more, 10 wt % or more, 16 wt % or more, 23 wt % or more, 30 wt % or more, 38 wt % or more or 40 wt % or more, and 40 wt % or less, 37 wt % or less, 31 wt % or less, 26 wt % or less, 18 wt % or less, 11 wt % or less, 7 wt % or less or 5 wt % or less.

For example, when the blue complex powder is used in an amount of less than 5.0 wt % based on the total weight of the composition, it may be difficult to achieve a desired cosmetic effect. And, when it is used in an amount exceeding 40 wt %, formulation stability may be unsatisfactory.

And, for example, if the average diameter of the total particles (that constitute the powder) is 20 μm or greater, when the blue complex powder is used in an amount of 20 wt % or more in a twin cake formulation in the form of a compact powder, applicability, uniformity and shapability may be unsatisfactory and blurring may occur.

In this aspect, the composition for improving skin transparency may be a cosmetic composition, specifically a cosmetic composition for makeup.

For example, the cosmetic composition for makeup may be in the form of a makeup primer, a makeup cosmetic, a foundation, a BB cream, a functional lotion, an eye shadow, etc. However, the formulation is not particularly limited and may be selected adequately depending on purposes.

In this aspect, the cosmetic composition may be for base makeup, although not being limited thereto.

In the present disclosure, a “cosmetic composition for base makeup” may mean a cosmetic composition which is used mainly to express clear skin by covering freckles, wrinkles, moles, etc. of the face. For example, it may be in the form of a liquid foundation, a cream foundation, a powder foundation, a two-way cake, a BB cream or a pact, although not being limited thereto.

When the formulation of the present disclosure is a paste, a cream or a gel, animal oil, vegetable oil, wax, paraffin, starch, tragacanth, cellulose derivatives, polyethylene glycol, silicone, bentonite, silica, talc, zinc oxide, etc. may be used as a carrier component.

When the formulation of the present disclosure is a powder or a spray, lactose, talc, silica, aluminum hydroxide, calcium silicate or polyamide powder may be used as a carrier component. In particular, when the formulation is a spray, it may further include a propellant such as chlorofluorohydrocarbon, propane/butane or dimethyl ether.

In an aspect of the present disclosure, the cosmetic composition may include various appropriate matrices and additives if necessary, and the kind and amount thereof can be easily determined by those skilled in the art. It may include allowable additives if necessary. For example, preservatives, pigments, additives, etc. commonly used in the art may be further included.

Specifically, phenoxyethanol, 1,2-hexanediol, etc. may be used as a preservative and a synthetic flavor may be used.

However, the additionally added components are not limited to those described above and any component may be added as long as it does not negatively affect the purpose and effect of the present disclosure.

Hereinafter, the present disclosure will be described in detail through examples. However, the following examples are for illustrative purposes only and it will be apparent to those of ordinary skill in the art that the scope of the present disclosure is not limited by the examples.

### [Example 1] Preparation of Twin Cake Composition Including Blue Complex Powder

A twin cake composition in the form of a compact powder was prepared according to a commonly employed method as described in Table 1. Flamenco Silk Blue (particle diameter: 1-16 μm) available from Badische Anilin & Soda-Fabrik (BASF) in Germany was used as a fine powder exhibiting blue color.

### [Comparative Example 1] Preparation of Twin Cake Composition with No Additive

A twin cake composition (Comparative Example 1) was prepared as described in Table 1.

### [Comparative Example 2] Preparation of Twin Cake Composition Including Normal Pearl Powder

A twin cake composition including a normal pearl powder (Comparative Example 2) was prepared as described in Table 1. Flamenco Blue (particle size distribution: 9-40 μm) available from BASF was used as a normal pearl powder.

### [Comparative Example 3] Preparation of Twin Cake Composition Including Blue Pigment

A twin cake composition including a blue pigment (Comparative Example 3) composition was prepared as described in Table 1. Ultramarine Blue available from Sun Chemical in the United Kingdom was used as a pigment. When the content of the blue pigment in the twin cake composition was the same as in Example 1, the composition could not function as a twin cake (due to turbidity and rough touch).

TABLE 1

| | | Comparative | Comparative | Comparative |
|---|---|---|---|---|
| Components | Example 1 | Example 1 | Example 2 | Example 3 |
| Talc | Balance | Balance | Balance | Balance |
| Sericite | 15.0 | 15.0 | 15.0 | 15.0 |
| Silica | 10.0 | 10.0 | 10.0 | 10.0 |
| Titanium dioxide | 10.0 | 10.0 | 10.0 | 10.0 |
| Zinc oxide | 3.0 | 3.0 | 3.0 | 3.0 |
| Fine pearl powder (blue) | 10.0 | — | — | — |
| Normal pearl powder (blue) | — | — | 10.0 | — |
| Ultramarine Blue | — | — | — | 0.2 |
| Iron oxide | 2.0 | 2.0 | 2.0 | 2.0 |
| Mineral oil | 7.0 | 7.0 | 7.0 | 7.0 |
| Preservative | 0.3 | 0.3 | 0.3 | 0.3 |

### [Example 2] Comparison of Reflection Spectra

To compare natural reflection of blue light by the compositions of Example 1, Comparative Example 1 and Comparative Example 3, reflection spectra were measured using a spectrophotometer after applying each composition on the face. Relative reflectivity was measured as compared to the reflectivity when nothing was applied on the face as 0.

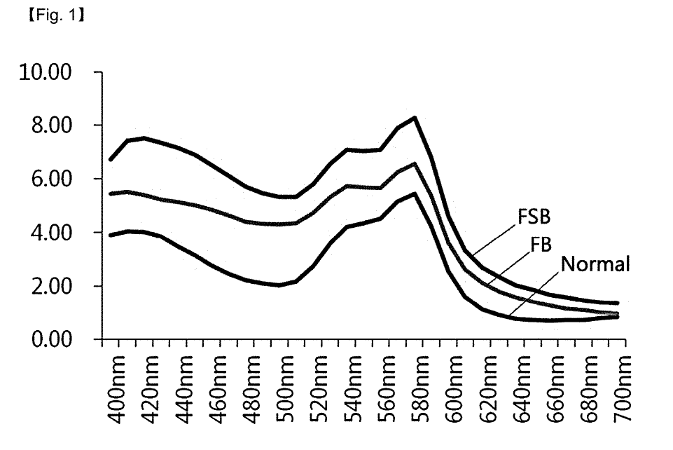

As a result (FIG. 1), the twin cake composition including blue complex powder showed relatively higher reflectivity at 400-450 nm as compared to Comparative Examples 1 and 3.

### [Example 3] Evaluation of Usability by Panels

The usability of the twin cake composition including blue complex powder (Example 1) and the compositions of Comparative Example 1-3 was evaluated in terms of transparency, gloss, naturalness and uniformity. The panels consisted of 20 women in their 20s and 30s, who applied each twin cake composition on the face and evaluated transparency, gloss, naturalness and uniformity before and after the application. As a result, the twin cake composition including blue complex powder was superior in all the four items, as shown in Table 2.

In particular, the composition of Comparative Example 2 showed decrease in transparency, gloss, naturalness and uniformity during the preparation of the twin cake because the pearl powder was broken during pulverization and mixing.

TABLE 2

| | Natural- | | Trans- | |
|---|---|---|---|---|
| | ness | Gloss | parency | Uniformity |
| Example 1 | ⊚ | ◯ | ◯ | ◯ |
| Comparative Example 1 | ◯ | Δ | Δ | ◯ |
| Comparative Example 2 | ◯ | ◯ | ◯ | Δ |
| Comparative Example 3 | Δ | Δ | Δ | ◯ |

(⊚: very superior, ◯: superior, Δ: moderate, X: poor)

1. A composition for improving skin transparency, which comprises a blue complex powder as an active ingredient,
wherein
the complex powder comprises a flat-shaped fine powder and a coating layer coated on the flat-shaped fine powder,
the flat-shaped fine powder is one or more selected from a group consisting of mica, synthetic mica, alumina, borosilicate, talc and sericite,
the coating layer comprises one or more selected from a group consisting of titanium dioxide and tin oxide, and
90% or more of the particle constituting the complex powder has a size within a range of 1-16 μm.

2. The composition for improving skin transparency according to claim 1, wherein the blue complex powder reflects blue light having a wavelength of 390-500 nm.

3. The composition for improving skin transparency according to claim 1, wherein the blue complex powder reflects blue light having a wavelength of 400-450 nm.

4. The composition for improving skin transparency according to claim 1, wherein the coating layer is comprised in an amount of 2-60 wt % based on the total weight of the blue complex powder.

5. The composition for improving skin transparency according to claim 1, wherein the composition comprises 5-40 wt % of the blue complex powder based on the total weight of the composition.

6. The composition for improving skin transparency according to claim 1, wherein the composition is a cosmetic composition.

7. The composition for improving skin transparency according to claim 6, wherein the cosmetic composition is for makeup.

8. The composition for improving skin transparency according to claim 7, wherein the cosmetic composition is for base makeup.

9. A method for improving skin transparency, which comprises applying an effective amount of a blue complex powder to a subject in need thereof,
wherein
the complex powder comprises a flat-shaped fine powder and a coating layer coated on the flat-shaped fine powder,
the flat-shaped fine powder is one or more selected from a group consisting of mica, synthetic mica, alumina, borosilicate, talc and sericite,
the coating layer comprises one or more selected from a group consisting of titanium dioxide and tin oxide, and
90% or more of the particle constituting the complex powder has a size within a range of 1-16 μm.

10. The method according to claim 9, wherein the blue complex powder reflects blue light having a wavelength of 390-500 nm.

11. The method according to claim 9, wherein the blue complex powder reflects blue light having a wavelength of 400-450 nm.

12. The method according to claim 9, wherein the coating layer is comprised in an amount of 2-60 wt % based on the total weight of the blue complex powder.

13. The method according to claim 9, wherein the blue complex powder is applied in a form of a composition, wherein the composition comprises 5-40 wt % of the blue complex powder based on the total weight of the composition.

14. The method according to claim 13, wherein the composition is a cosmetic composition.

15. The method according to claim 14, wherein the cosmetic composition is for makeup.

16. The method according to claim 15, wherein the cosmetic composition is for base makeup.
